(19) United States
(12) Patent Application Publication
(10) Pub. No.: US 2003/0176868 A1
(43) Pub. Date: Sep. 18, 2003

(54) Long bone reaming apparatus and method

(76) Inventor: John R. Pepper, Cheshire, CT (US)

Correspondence Address: JOHN R. PEPPER, 224 BEACON HILL DRIVE, CHESHIRE, CT, 06410, US

(21) Appl. No.: 10/369,737

(22) Filed: Feb. 21, 2003

Related U.S. Application Data
(60) Provisional application No. 60/358,587, filed on Feb. 22, 2002.

(51) Int. Cl. A61B 17/16

(52) U.S. Cl. 606/80

(57) ABSTRACT

A slender rod is used to guide a circular cutter thought the intramedullary canal of a long bone. Loading and unloading of the cutter is done quickly by having a reduced cross section near one end of the rod. The cutter has a corresponding slot radially extending from its center. The cutter can be disengaged from a driving shaft without disengaging the driving shaft from the rod, eliminating a difficult realignment process, making the whole cutting process faster. The slot will not interfere with the cutting operation, nor allow the cutter to break free. The reduced section of the rod can be modular and replaceable.

### CROSS REFERENCE TO RELATED APPLICATION

This application claims the benefits of Provisional Patent Application Ser. No. 60/358,587 filed Feb. 22, 2002

### FEDERALLY SPONSORED RESEARCH

This was not federally sponsored research.

### BACKGROUND OF THE INVENTION

The treatment of long bone fractures typically involves internal stabilization. The benefits over external stabilization casting traction and plating include, better alignment, less invasive procedure, faster weight bearing, faster recovery, and less blood loss. An intramedullary nail or rod is a cylindrical usually hollow rod inserted in the center of the intramedullary canal or marrow cavity. The rod is usually titanium or stainless steel and is strong enough to support the bone loads during the bones healing process. The bones that are fixed in this manner are the femur, tibia, humerus and radius an ulna. Nails are typically generally circular in cross section or have a shape nearly circular.

One step in the surgical technique is the preparation of the canal. It varies in shape depending upon the position along the bone axis. The center of the bone is called the isthmus, which is a narrowing of the canal. This is especially true in the femur where in order to carry the body weight, the bone gets quite thick in its center.

A nail must be sized to carry the body weight. In order to get present day nails large enough to carry these loads, typical one and half to three times body weight, the rod must be larger than the canal. What is presently done; is to enlarge the canal. This is done is the following steps. A guide rod is inserted in the canal over it entire length. It is usually two to four millimeters in diameter and is 500 to 1000 millimeters long. It serves several purposes; first it is used to align the fragments of bone. A surgeon will use the guide rod to thread the segments through their canals. Secondly the guide rod is used to guide cutters through the canal to enlarge it to accept the nail.

The reaming process has several components. A drill with a hole through is driving axis is used to supply power. Bone is difficult to cut, especially in young males, who are frequently the fracture patient. The drill is either pneumatic or battery powered. It can impart a high torque on all driving components in order to ream the bone. As the canal of the bone is curved, a flexible shaft is used to couple the drill and the reamer. The flexible shaft is usually about 450 millimeters long and eight to twelve millimeters in diameter. It is cannulated, and has a hole along its length slightly larger than the guide rod. It has a connection means on one end for the drill and on the other for connection with the reamer head (cutters). The connection to the cutter can be a radial or side loading dovetail. It can also be an axial loading quick connect that uses the guide rod as a locking means.

The reaming set has a set of cutter that increase in diameter in increments of one half to one millimeter. A set of reamer heads for the femur may have reamer heads from nine to fourteen millimeters. The reamers are typical less than three centimeters in length, as longer cutter could not follow the curve of the bone. The reaming is done to one half to one millimeter over the selected nail size so it is easy to insert.

The reaming is done by attaching a reamer had to the flex shaft and then threading these two parts over the end of the guide rod. They are advanced to bone, cut the bone and are withdrawn. The cutter is pulled back off the guide rod and then disconnected from the flexible shaft by moving the cutter radially. The next size reamer head is attached to the flexible shaft radially, and then the assembly is axially threaded onto the guide rod and the process is repeated until the desired cavity size is prepared.

It is common for six to ten reaming steps to be needed to make the canal of sufficient size to accept a nail. This is a slow and tedious part of the surgery. The difficulty arises in that the guide rod is very long and is only slightly more rigid than a coat hanger. The flexible shaft is also pretty flimsy and almost as long. The guide rod's long length outside the body make it a hard target to hit as a three millimeter rod must be axially aligned with a three millimeter nominal sized hole in the flexible shaft with only about a quarter millimeter of tolerance. Two long flexible parts must be perfectly axially aligned in order to be threaded.

These steps show how it is done. The surgeon must hold the dovetail style cutter to the flexible shaft while trying to do the threading process. At the same time the drill must be supported. This process usually required at least three hands. One to hold the drill, one to hold the flex shaft reamer connection and one to hold the guide rod steady so they can be axially aligned. As the surgeon's gloves at this time are wet with blood and fat from the canal, they are very slick and only makes this more difficult. Some surgeons that are very skilled can hold the drill with one hand, and use the other to hold the reamer to the shaft, and capture the bouncing guide rod, then align it all together one handedly. Few indeed are those who have this type of dexterity.

The axially loading flex shaft/cutter connections do load these two parts together faster and may hold them together on their own, however they still require three hands to re thread the cutter over the guide rod. The most difficult part of the case is the threading the cutters on the guide rod and it must be done many times.

If the reamer head is dropped from the hand onto the floor during this process, it must be sterilized delaying the case even further. Clearly reaming is a frustrating part of the long bone fracture fixation procedure, and it is no wonder it is left to residents and those in their medial training to do this tedious task.

### BREIF SUMMARY OF THE INVENTION

The present invention allows changing of the reamer heads without removal of the flexible shaft from the guide rod.

### BRIEF DESCRIPTION OF THE DRAWINGS

FIG. 1A is a perspective view of the prior art showing a long bone (femur), drill, flexible reamer shaft (shortened and simplified) guide rod (shortened), and cylindrical cutter, just prior to reaming the bone.

FIG. 2 is a perspective view of the prior art showing the reamer assembly disengaged from the guide rod in preparation for reamer exchange.

FIG. 3A is a perspective view of the prior art, showing the radial engagement of the reamer and dovetail reamer connection, off the guide rod.

FIG. 3B is a close up perspective view of the prior art, showing the radial engagement of the reamer and dovetail reamer connection, off the guide rod.

FIG. 4 is a perspective view of the prior art, a guide rod with end stop, straight, shown shortened for clarity.

FIG. 5A is a perspective view of the prior art, a ten mm diameter reamer head with the helical cutting teeth omitted for clarity.

FIG. 5B is a perspective view of the prior art, a twelve mm diameter reamer head with the helical cutting teeth omitted for clarity.

FIG. 5C is a perspective view of the prior art, a fourteen mm diameter reamer head with the helical cutting teeth omitted for clarity.

FIG. 6 is a perspective view of an embodiment, the one piece, multiple cross section shaft of guide rod, with the larger cross section portion of the shaft shortened for clarity.

FIG. 7A is a perspective view of another embodiment, a multi-piece, multiple cross section shaft guide rod, assembled, larger cross section portion of the shaft shortened for clarity.

FIG. 7B is a plan view of a cross section of multiple-piece rod, larger cross portion of the section shaft shortened for clarity.

FIG. 7C is a detail of the plan view of threaded connection of the multiple piece rod.

FIG. 8A is a perspective view of an alternate embodiment of guide rod with parallel cuts, cut away from end, with the larger cross section portion of the shaft shortened for clarity. Ball end omitted.

FIG. 8B is a plan view of an alternate embodiment of guide rod with parallel cuts, cut away from end.

FIG. 9A is a perspective view of an alternate embodiment of guide rod with a polygon cross section, cut away from end.

FIG. 9B is a perspective view of an alternate embodiment of guide rod with a polygon cross section, cut away from end.

FIG. 10 is a perspective view of the embodiment showing positioning of cutter, flexible shaft, and guide rod components prior to radial loading. The large section of the guide rod is shown shortened for drawing clarity.

FIGS. 11A-F are perspective views of an embodiment showing progressive radial engagement of the cutter guided by the small section and the connector on the shaft.

FIGS. 12A-C are perspective views of the components in FIG. 10 to illustrate the advancement of cutter and flexible shaft from the small cross to the large cross section of the rod.

FIGS. 13A-C are perspective views of the components in FIG. 10 to illustrate the advancement of cutter and flexible shaft from the small cross to the large cross section of the rod rotated to show the junction position.

FIGS. 14A-B are perspective views of the embodiment of FIG. 10 to show the radial slot guiding the small cross section.

FIGS. 15A-B are perspective views of the embodiment of FIG. 8A with the cutter and flexible shaft in alignment for radial engagement.

FIG. 16A is a plan view of the loading of cutter relative to intramedullary canal, guide rod assembly and femur.

FIG. 16B is a perspective view of the loading of cutter relative to intramedullary canal, guide rod assembly and femur.

FIG. 17A is a perspective view of the cutter on the guide rod, attached to the flexible shaft, ready to ream the canal.

FIG. 17B is a detailed view of the cutter of FIG. 17A.

FIG. 18 is a perspective view of the prior art cutter with cutting flutes.

FIG. 18B is a perspective view of the a cutter showing the cutting flutes and the radial slot.

FIG. 19A is a perspective view of small cross section rod with constant cross section.

FIG. 19B is a perspective detail view of the embodiment of FIG. 19A showing the locking thread and the leading alignment boss.

FIG. 20 is a perspective view of the main guide rod of large cross section with a threaded recess for the second, small section rod component.

FIG. 21 A is a perspective view of a two piece embodiment with dual diameters with a flexible shaft alignment boss on the small section component shown assembled.

FIG. 21B is a plan view of a two piece embodiment with dual diameters with a flexible shaft alignment boss.

FIG. 22 is a perspective view of the cutter on the two piece embodiments with a flexible shaft alignment boss.

FIG. 23 is a perspective view of the two piece embodiment with a flexible shaft alignment boss and flange adjacent to the locking thread.

FIG. 24 is a plan view of the small section with the flexible shaft alignment segment, and the flange adjacent to the locking thread.

FIG. 25 is a perspective view of the embodiment shown in FIG. 24.

FIG. 26 is a perspective view of a one piece guide rod with integral small section and upper large section for alignment.

### DETAILED DESCRIPTION OF THE INVENTION

The present invention is intended to alleviate the drawbacks of the conventional axially loaded intramedullary reamer used in long bone fixation surgery. An object of this invention is to provide a radially loading reamer that does not necessitate separation of the reamer shaft and the guide rod.

To accomplish the above recited object, the present invention consists of a long slender rod to fit the intramedullary canal of a long bone with a plurality of cross sections areas is used to guide reamers (cutters) within the intramedullary canal of the bone. The cutters are conventional reaming heads with the addition of a radial slot extending from the central bore.

The preferred embodiment has two main shaft cross sections, and both sections are circular.

The guide rod can be constructed from one component. The advantage to one component is that is preassembled, however the small diameter shaft can bend during manipulation prior to reaming. Straightening a bent rod intra-operatively can be difficult.

Alternatively the rod can be composed of a more than one segment. One component looks externally like a conventional guide rod with an engagement means on the end opposite the ball end. The engagement means could be contained within an internal cavity, a thread. The second component is a smaller cross section rod, or loading section. The preferred embodiment would be a round section.

The small section could have an engagement means on it. It can be a friction fit into a smooth bore. The preferred embodiment is a thread. The two components could line up axially and lock together. They would then function like the unitary component device. The small component could be added after the fracture manipulation is complete lessening the chance for a bent small section.

The small section rod could be a commonly used orthopaedic wire, or Kirschner wire (K wire), used for a multitude of procedures.

The length of the small section is has to be slightly longer than the cutter. It could be much longer than the cutter as K wires can be over ten centimeters long. The preferred embodiment for ease of loading, flexible shaft retention would be approximately five centimeters. That typically would allow a few centimeters of small shaft to extend beyond the cutter to hold the flexible shaft in place.

The locking means between each rod segment holds the small section on while the flexible reamer is being moved back and forth. There is some friction between the guide rod and inner portion of the flexible reamer. Axial resistance to the motion of the small relative to large segment could be done with a threaded connection. The reamers tend to run in one direction only, so a standard right hand thread would tend to self tighten during operation.

Typical sizes of the rod main portion would be from two and four millimeters in diameter, and the smaller cross section is between one to two millimeters in diameter. The smaller section would typical have a size to be fifty percent of the larger.

The small cross section can be made by removal of material from a conventional guide rod. This can be done in one or more planes, so the cross section can form a polygon. These cuts can be adjacent to the end of the guide rod, or they can be located a short distance from the end. The later embodiment allows a full section of guide rod to center the flexible shaft and its dovetail (or equivalent locking means) over the rod to further speed reamer loading. This method is somewhat more difficult to manufacture, as working with a long flexible rod is difficult.

The loading section could have both small and large sections, allowing radially loading while maintaining the centering of a full section on the flexible shaft bearing surface, at the same time, keeping the economy of a constant section main guide rod.

This embodiment of the lading section is also replaceable to reduce bending risk.

Another loading section has two diameter section equal to the main guide rod flanking a smaller loading section. This allows the advanced centering, radially loading of the previous embodiments, and provides an abutment surface to stop the thread engagement, stiffening the junction between the main guide rod and the loader section.

The loading section could have a tapered approach to facilitate loading of eh reamer shaft initially.

The small cross section of the above embodiments is long enough to clear the length of the reamer, approximately three centimeters. In cases where the small section is not backed up with a larger cross section, the shorter section can be extended to maintain the flexible shaft on the rod. An overlap of two centimeters is adequate to keep the flexible shaft in place. The length of the straight short portion could then be five centimeters.

The main portion of the two piece embodiment would be from 500 to 1000 millimeters long. This depends on the bone that is being reamed. Generally the rod is about twice the length of the canal of the bone.

The main portion of the two piece assembly has a stop on the end going into the canal to prevent reamer dissociation. The unitary guide rod has a stop to prevent reamer dissociation also.

The reamer or cylindrical cutter enlarges the intramedullary canal by cutting a round hole. This hole will provide means to place an intramedullary rod. The cutters generally are tapered or barrel shape to follow previous cutters, and have a good cutting action. The radial slot is cut from the central bore to the outer edge. It is located to minimize the disturbance to the cutting edges of the flutes. Flutes that must be divided are done so such that there are no weak sections or unintended sharp edges. The slot is slightly wider than the small section of the rod. The reamer head can then slide on and off of the rod. When the reamer is advanced onto the main portion of the rod, it spins freely and can not move radially because the slot is smaller than the guide rod at that portion. At this point it functions like a typical reamer.

When the reamer is to be exchanged, the flexible drive shaft draws it back out of the canal and up the rod so the reamer head is over the smaller section. The cutter is slid off the guide rod along a radial path.

When the loading section is a constant diameter (K wire) the flex shaft is held in place and provides some movement between the rod and flexible shaft connector.

With the multiple diameter loading section, the larger upper section perfectly centers the flexible shaft so that no alignment is needed. The round cross section of the smaller section does not require special alignment either. The only alignment necessary is that of the reamer dovetail to the flexible shaft, which is as it is required on present reaming systems.

In the embodiment of a guide rod with the polygon shaped reduced section, the reamer engagement must be aligned with the polygon before the reamer can be loaded.

With all of the embodiments, once the smaller reamer is removed, the next sized reamer is placed over the small section, locked with the dovetail and advanced into the canal.

Another embodiment is for the transitions in guide rod diameters have tapers to make it easier for a reamer to go from one to another without getting caught. This can be adapted to all previous embodiments.

The extra section for the two piece can have driving means on one end to lock the threads in place and to remove it if need be. These can be a screw driver slot, external or internal polygon shape or a surface geometry such as a knurl.

In another embodiment of the loading rod, the tip adjacent to the thread has a diameter to facilitate centering within the thread, making the connection faster.

### REFERENCE NUMBER IN THE DRAWINGS

10 femur

20 drill

30 flexible reamer shaft

40 cutter

50 guide rod,

60 axial separation

70 dovetail

75 internal cannula

80 relief.

90 dovetail cavity

100 relief channel

110 cannulation

110 cutter cannula

120 stop or ball end

130 two cross sectioned shaft guide rod with ball end.

140 small cross section portion

150 junction

160 large cross section portion

170 small section

180 large section

190 threaded portion

200 threaded recess

210 rectangular

220 square

230 cutter

240 radial slot opening

250 bone's canal

260 cutting flutes

270 recesses.

280 alignment section

290 integral flexible shaft alignment section

300 large sections.

310 driving means

### DETAILED DESCRIPTION

For purposes of promoting an understanding of the principles of the invention, reference will now be made to the embodiments illustrated in the drawings and specific language will be used to describe the same. It will nevertheless be understood that no limitation of the scope of the invention is thereby intended, there being contemplated such alterations and modifications of the illustrated device, and such further applications of the principles of the invention as disclosed herein, as would normally occur to one skilled in the art to which the invention pertains.

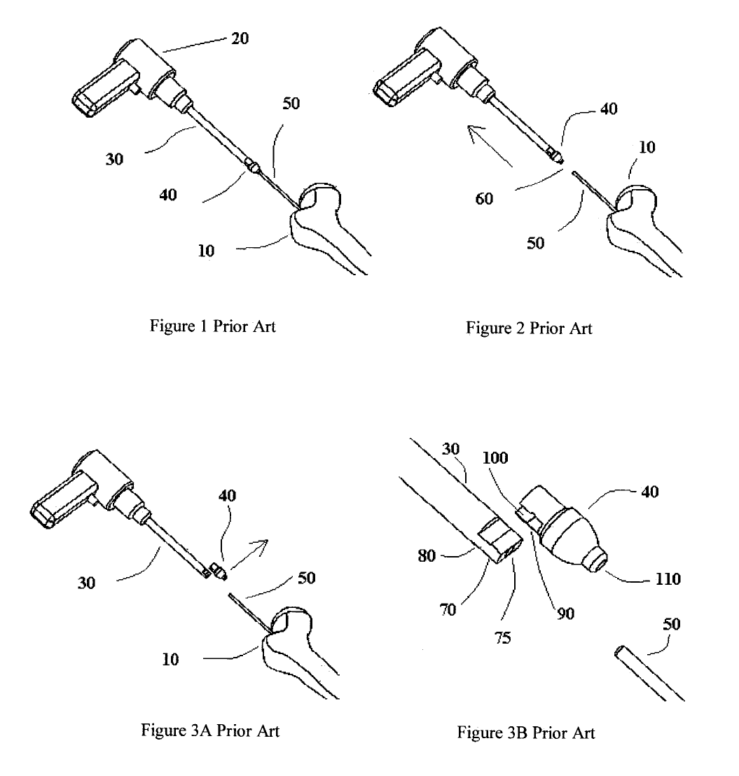

FIG. 1 shows the prior art. A long bone, in this case a femur 10, is reamed with a drill 20 coupled to a flexible reamer shaft 30 and a cutter 40. This assembly is slid over a guide rod, 50 into the intramedullary canal. The flexible shaft 30 and the cutter 40 are locked together so they rotate at the same speed. The drill drives these two components into the bone 10, to create a cylindrical cavity for a fracture fixing rod.

FIG. 2 is of prior art showing the axial separation 60 of the cutter/flexible shaft/drill and the guide rod 50 in preparation for reamer exchange.

FIG. 3 is of prior art showing the radial loading of the cutter 40 onto the flexible shaft 30.

FIG. 3B is of prior art showing details of a typical flexible shaft and reamer connection. The flexible shaft has a dovetail 70 adjacent to a relief 80. The cuter has a corresponding dovetail cavity 90 and smaller relief channel 100. The cuter has a cannulation 110 extending through its length. The cannulation is slightly larger than the guide rod 50. The flexible shaft 30 has an internal cannula 75 that is the same size as the cutter cannula 110 and both of these are slightly larger than the guide rod 50, so everything will easily rotate about the guide rod 50, when powered by the drill.

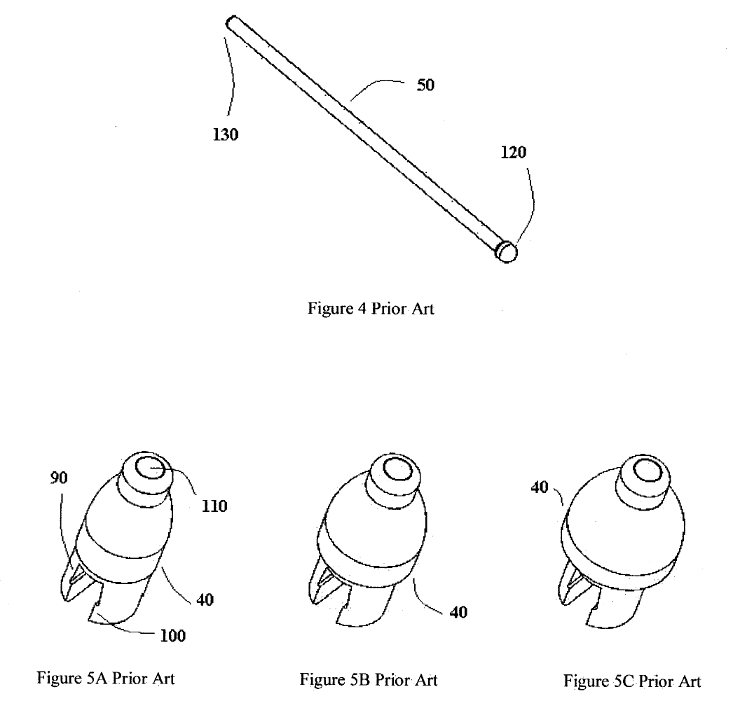

FIG. 4 shows the prior art guide rod 50. It is typically a solid rod with a stop or ball end 120. The ball end 120 is larger than the cutter cannulation 110 and will not allow the cutter to pass. This keeps the cutter from coming off inside the bone. The ball is typical welded or silver soldered onto the rod. The rod can be from 300 to 1000 millimeters long.

FIGS. 5A,B,C show the prior art. The cutters have identical dovetail cavity and reliefs, but the main diameter increases.

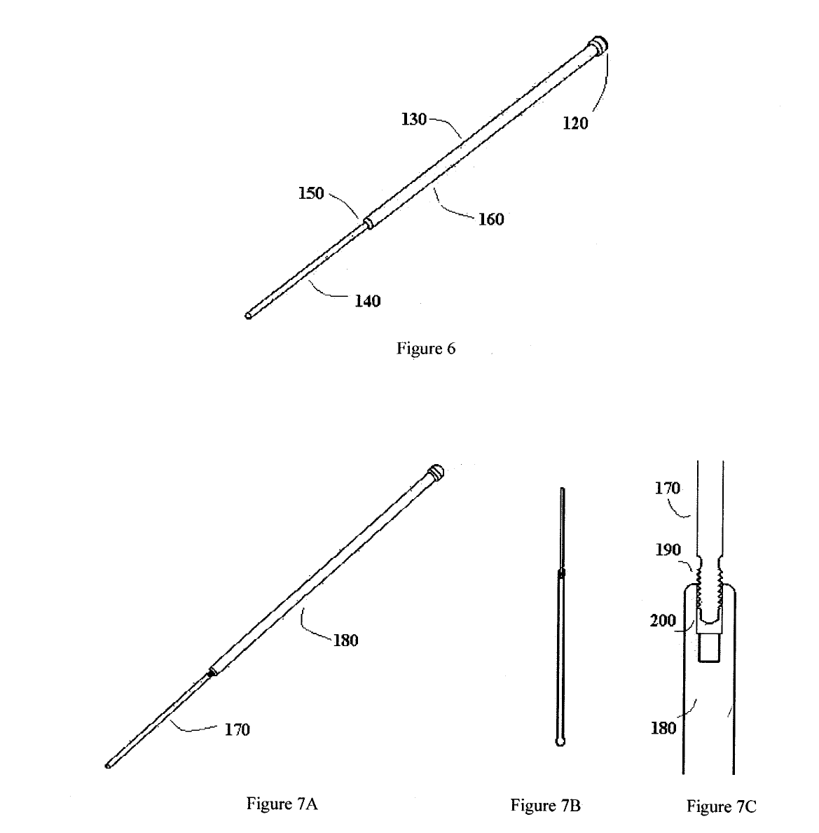

FIG. 6 shows the two cross sectioned shaft of rod with ball end shows one embodiment of the invention 130. It consists of a large cross section portion 160, a small cross section portion 140 and a ball end 120. The junction 150 is tapered to facilitate cutter transfer. The components line up along their axes. The large cross section 160 and the ball end 120 are similar in shape to the prior art.

FIGS. 7A+B shows the inventive guide rod can consist of more than one component. The small section 170 can be exclusively on one component, and the large section on another 180. The two components are joined together to functional as one with a connection.

A threaded connector is shown in FIG. 7C. The small section rod component 170 has a threaded portion on one end 190, mates with a threaded recess 200 in the large section component 180.

The cross section of the smaller section 170 is shown as circular.

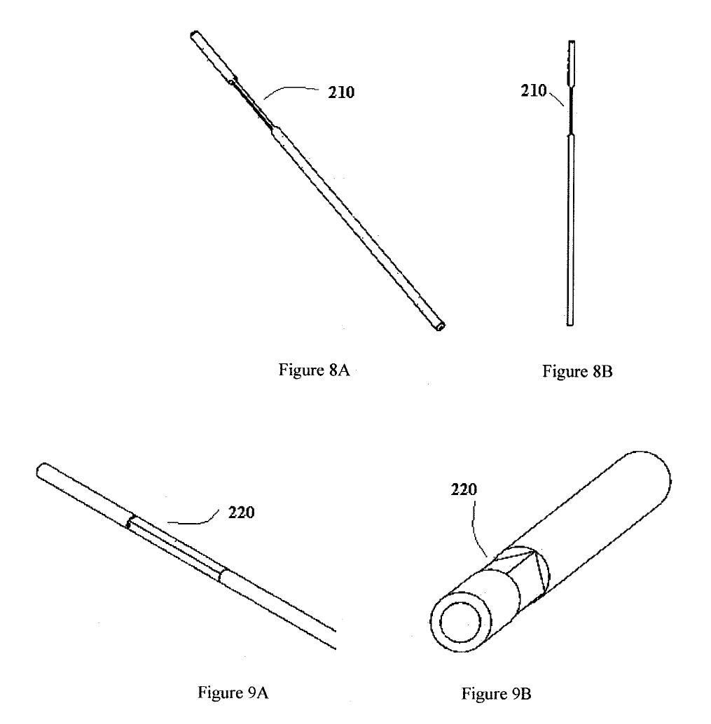

The smaller cross section can be non circular and can be generally rectangular 210 or square 220 as shown in FIGS. 8A-D.

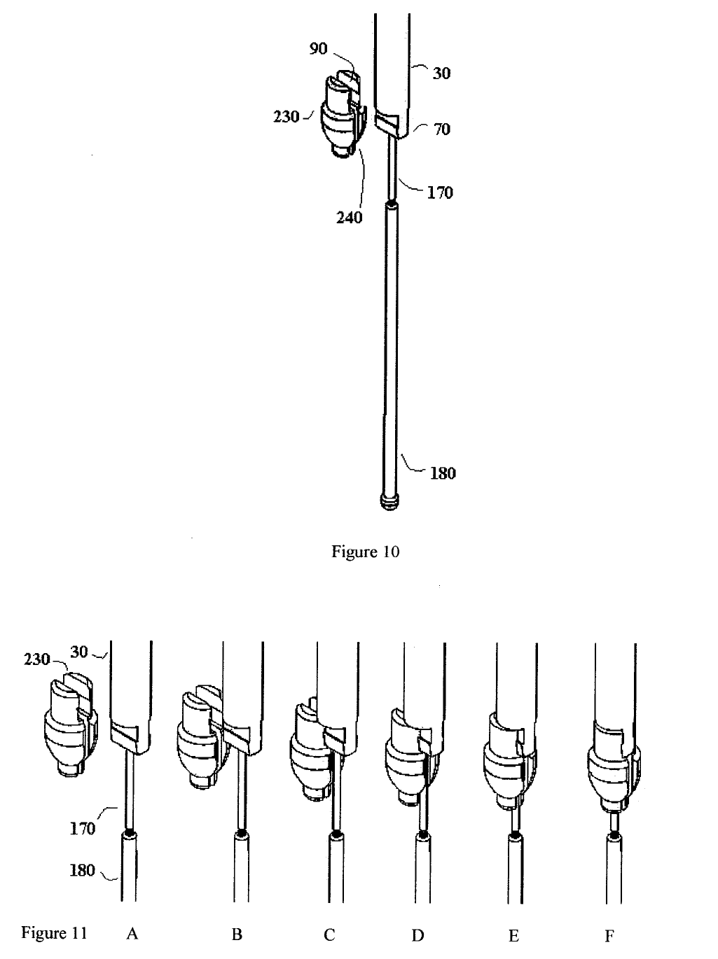

FIG. 10 shows a flexible shaft 30 goes over the guide rod small section 170. The inventive cutter 230 is positioned so the dovetail locking feature 90 is aligned with the mating geometry on the flexible shaft 70 and the inventive radial slot opening 240 is directed toward the small section.

FIGS. 11A-F shows the cutter 230 is advanced radially toward the center of the guide rod small section 170.

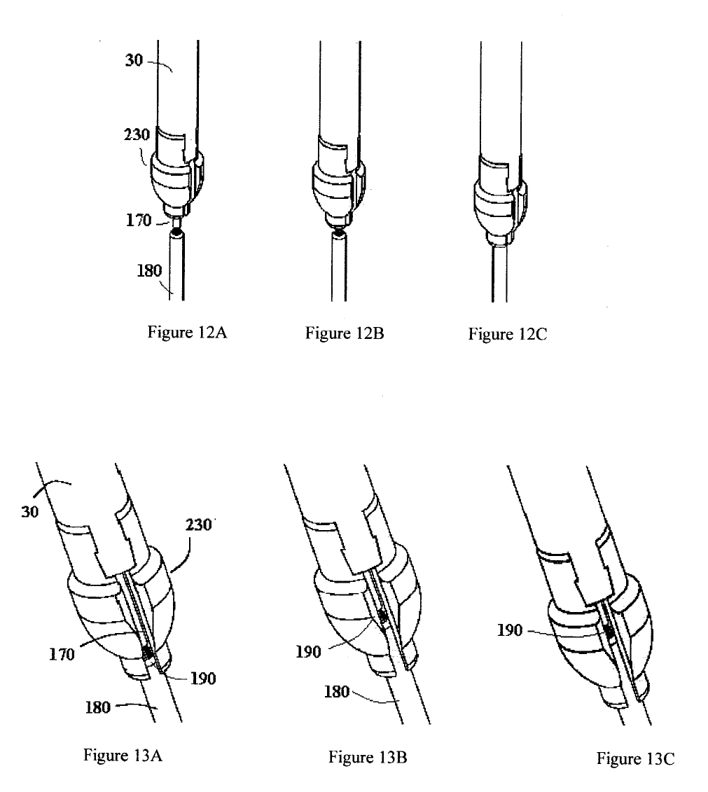

FIGS. 12A-C show the cutter 230 and flexible shaft 30 are advanced axially down the guide rod 180.

FIGS. 13A-C show the smaller section of the guide rod 170 going into the flexible shaft 30 and the inventive cutter cannula 10 fits over the guide rod large section 180.

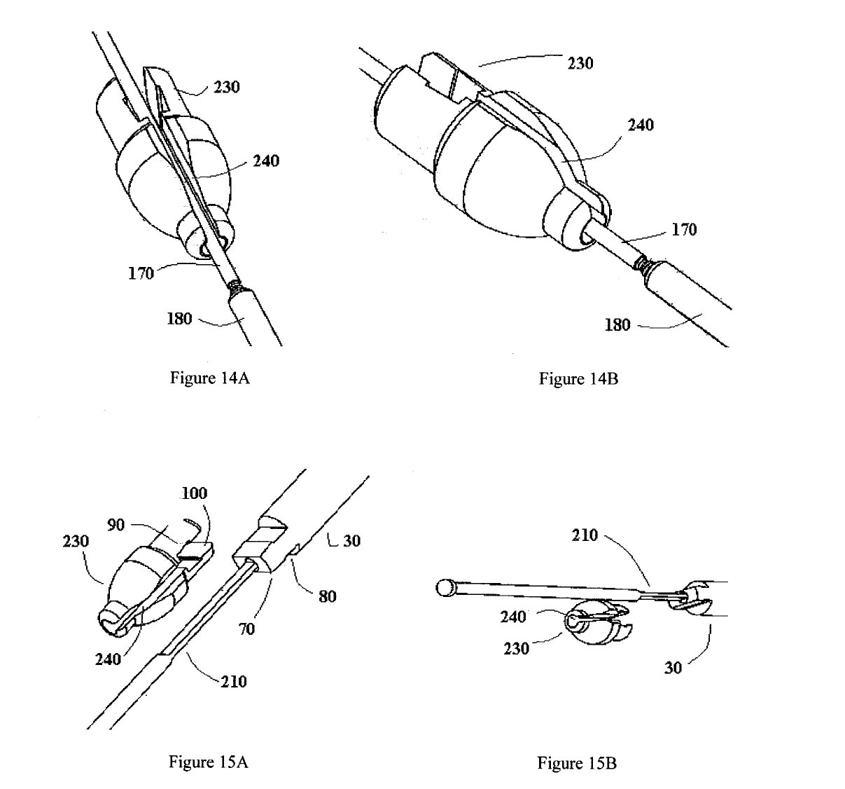

FIGS. 14A+B show the detail of the cutter retention means. The small section 170 can pass through the radial slot 240. The large section 180 can not, and the cutter 230 can freely rotate on the main rod section 180.

FIGS. 15A+B show the non round smaller section guide rod 210 align with the flexible shaft dovetail 80. The geometry on the guide rod 210 must be aligned with the slot 240 in the cutter 230 to advance the cutter radially to the locking position.

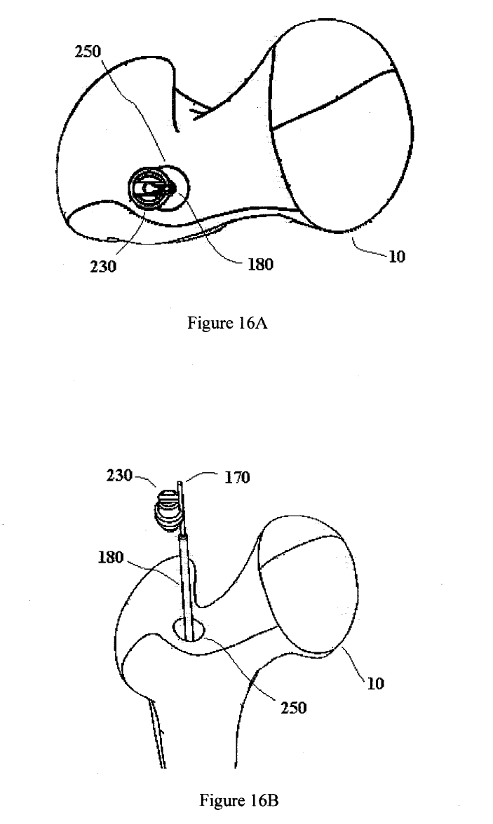

FIGS. 16A+B shows the loading of the cutter 230 is done adjacent to the bone 10. Most of the guide rod large section 180 is in the bone's canal 250 while loading occurs.

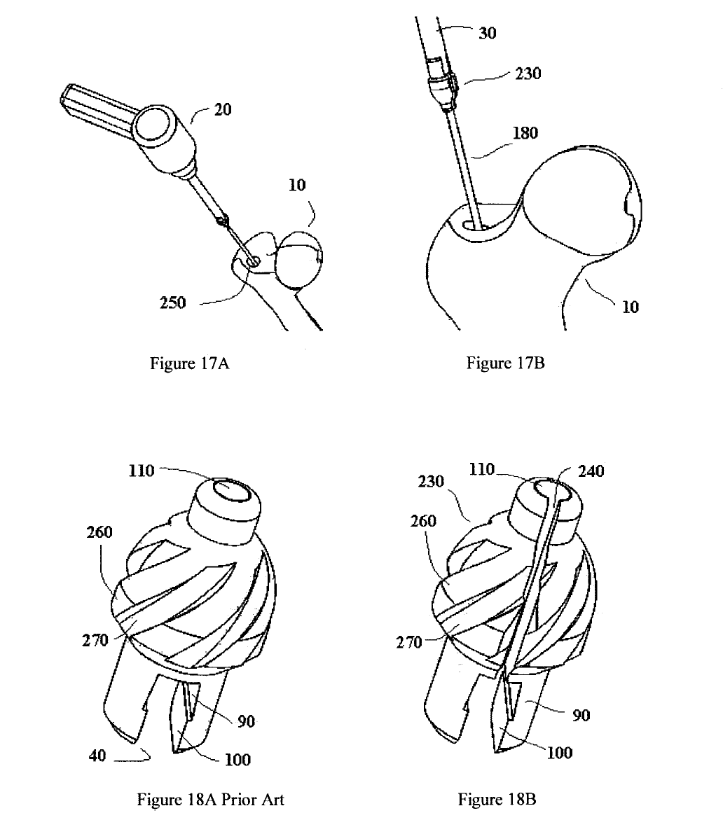

FIGS. 17A+B shows the cutter 230 with the radial slots 240 is positioned ready to go into the intramedullary canal 250. The drill 20 and flexible shaft 30 advance the cutter 230 over the guide rod large section 180 into the canal 250.

FIG. 18A shows a conventional cutter 40 with cutting flutes 260 and recesses 270 shown.

FIG. 18B shows the inventive cutter 230 with cutting flutes 260, recesses 270 and the inventive radial slot 240 shown. The cannulation 110 of the cutter is the same as the one shown in the prior art cutter 40. The flexible shaft retention means, shown here as a dovetail interlock, 90 and 100, are also the same as the prior art.

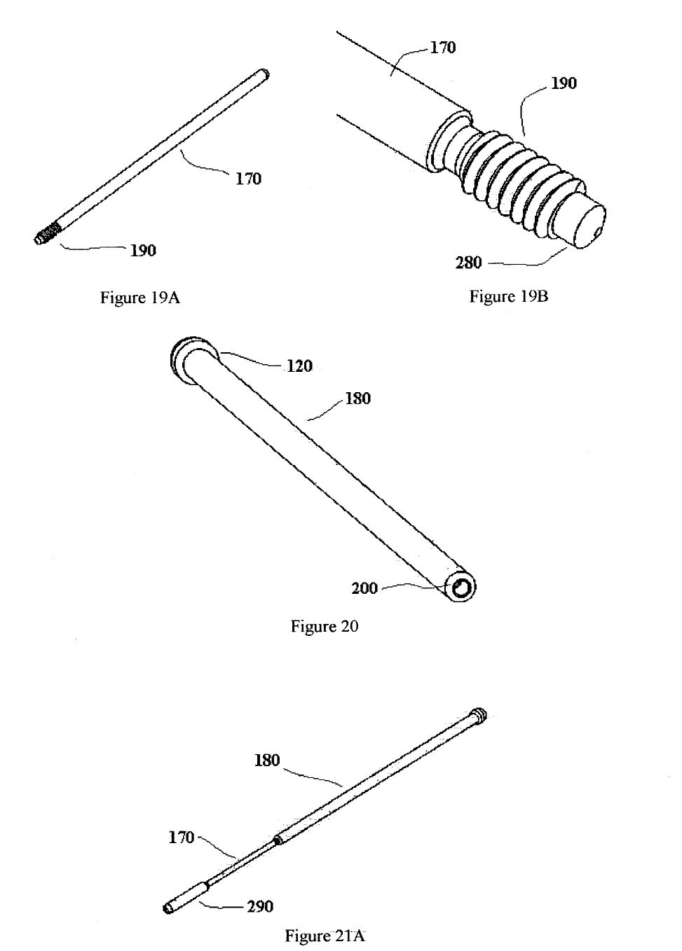

FIG. 19A shows the single cross section small section component 170 of the guide rod assembly. The cross section is circular of maximum stiffness and ease of manufacture.

FIG. 19B shows a detail of the small cross section 170. A thread 190 is used for joining the component of the guide rod, with an alignment section 280 to align and help start the threading process.

FIG. 20 shows the inventive large section guide rod component 180 with a connecter means 200, a threaded hole shown in the non ball end.

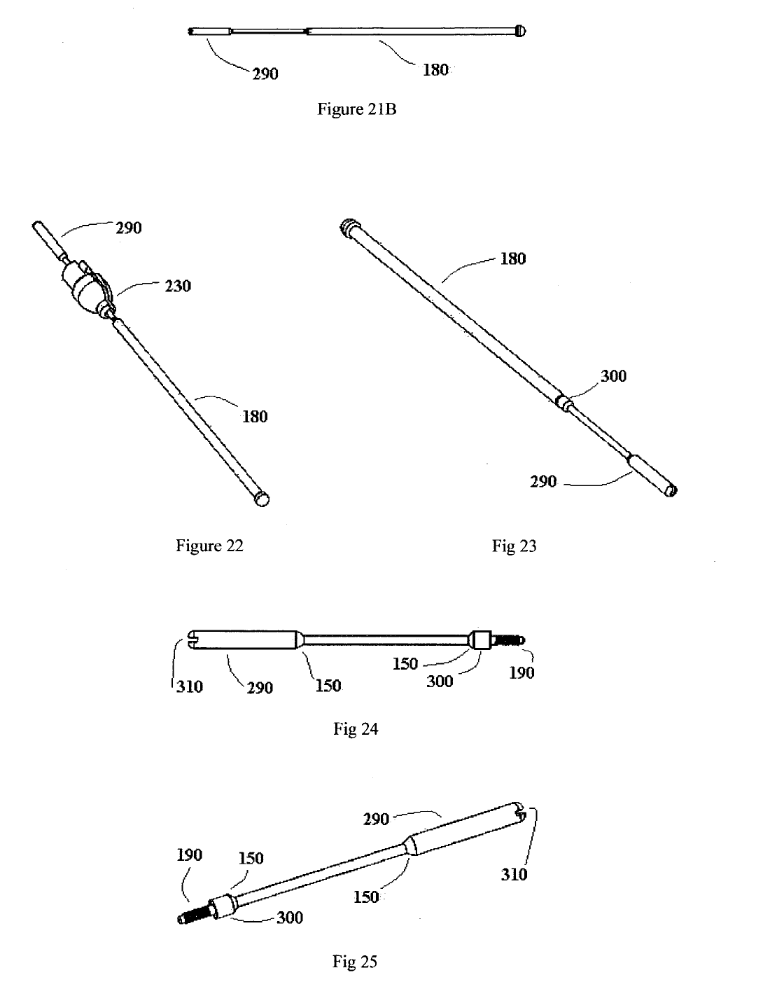

FIGS. 21A+B shows the inventive guide rod assembly with a small section segment with an integral flexible shaft alignment section 290. The flexible shaft alignment section has a diameter approximately the same diameter as the main guide rod. The internal cannula of the flexible shaft 75 is a slip fit over the guide rod 180. The flexible shaft alignment section 290 centers the dovetail of the cutter aligned with the dovetail of the flexible shaft so the radial slot does not require its own alignment.

FIG. 22 shows the cutter 230 relative to the small section and the flexible shaft alignment section 290.

FIG. 23 shows an embodiment of the small section guide rod component with two large sections adjacent 290 and 300. The large section 300 adjacent to the threads stabilizes the thread.

FIGS. 24 and 25 shows a driving means to engage the small diameter section with a driving tool 310. The driving means 310 is shown as a slot for a screw driver. The tapers 150 are to ease axial travel of the cutter.

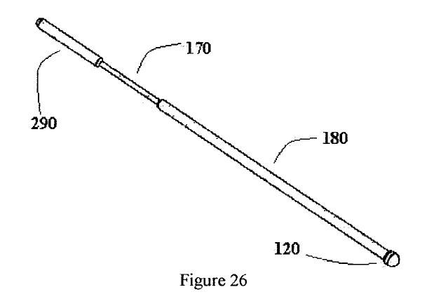

FIG. 26 shows an embodiment of the guide rod with the main section 180, the small section 170 and the alignment section 290 all in one piece.

I claim:

1. A long slender rod to fit the intramedullary canal of a long bone with a plurality of cross sectional areas to guide reamers within the canal of the bone.

2. The device of claim one where all the cross sections are circular.

3. The device of claim one where the rod is constructed from one component.

4. The device of claim one where the rod is assembled from at least two components.

5. The device of claim 4 where the components are engaged with a threaded connector.

6. The device of claim 2 where the larger cross section is between two and four millimeters in diameter.

7. The device of claim 2 where the smaller cross section is between one to two millimeters in diameter.

8. The device of claim one where the cross section of the rod varies by plurality of cuts.

9. The device of claim one where the larger cross section of the rod has a working length of approximately 500 to 1000 millimeters.

10. The device of claim one where the reduced cross section is between ten and one hundred millimeters long.

11. The device of claim four where one portion of the rod has both large and small sections.

12. The device of claim four with a small portion length of 1 to 40 mm and a large section length of 5 to 100 mm.

13. The device of claim one where the rod has one enlarged end to stop a cylindrical cutter from sliding off the end.

14. A cutter for creating a cylindrical hole with a central bore and a radial slot of a width smaller than the central bore diameter intersecting the central bore.

15. A method to ream the canal of a long bone where a multiple cross sectioned guide rod allows a slotted cutter to be engaged radially on one diameter and advanced onto a larger diameter such that free rotation is allowed without disengagement.

16. A method of claim 15 where the guide rod has means to adapt a smaller cross section rod to its free end.

17. The method of claim 15 where the radial loading of the cutter on the guide rod also locks the cutter to the driving means.
